(19) United States
(12) Patent Application Publication
(10) Pub. No.: US 2016/0256615 A1
(43) Pub. Date: Sep. 8, 2016

(54) MEDICAL FLUID COLLECTION SYSTEMS, DEVICES, AND METHODS

(71) Applicant: FLEXICARE MEDICAL LIMITED, Mountain Ash (GB)

(72) Inventor: Ghassem Poormand, Newport Coast, CA (US)

(21) Appl. No.: 14/638,866

(22) Filed: Mar. 4, 2015

(51) Int. Cl. A61M 1/00

(52) U.S. Cl. CPC A61M 1/0086; A61M 1/0049

(57) ABSTRACT

The disclosure herein provides system, methods, and devices for collection of fluids, for example, bodily fluid and other fluids. An aspiration and fluid collection device for collecting bodily fluid and other fluids comprises a cap which may be mounted on a compressible resilient body comprising a cavity for storage of collected bodily fluid and other fluids. The cap comprises an inlet port comprising a first one-way valve positioned to enable bodily fluid and other fluids to flow into the cavity through the inlet port, but not out of the cavity through the inlet port; and an outlet port comprising a gas permeable but liquid resistant membrane positioned to enable gas to be expelled from the cavity through the outlet port, but to resist bodily fluid and other fluids being expelled from the cavity through the outlet port. The outlet port further comprises a second one-way valve positioned to enable gas to be expelled from the cavity through the outlet port, but not into the cavity through the outlet port.

### BACKGROUND

1. Field

The disclosure relates generally to the field of medical devices, and more specifically to medical systems, devices, and methods for bodily fluid and other fluids suction and collection.

2. Description

Many medical procedures cause a patient to bleed, or to produce other excess bodily fluids and other fluids, for a period of time during the procedure and in recovery after the medical procedure. For example, rhinoplasty and sinus operations may result in a patient bleeding from the nasal cavity for an extended period of time after the procedure. Other examples include bleeding from post-operative wounds from mastectomy and abdominoplasty. Further, the patient may be required to remain at the medical facility until such bleeding has stopped, increasing costs of the medical procedure and inconveniencing the patient. Accordingly, it can be advantageous to have portable systems, devices, and methods for collecting the excess bodily fluid and other fluids to, among other things, enable a patient to be released earlier to reduce costs and decrease patient inconvenience.

### SUMMARY

The disclosure herein provides systems, methods, and devices for portable suction and collection of bodily fluid and other fluids.

According to some embodiments, an aspiration and fluid collection device for collecting bodily fluid and other fluids comprises a compressible resilient body comprising a cavity for storage of collected bodily fluid and other fluids; an inlet port comprising a first one-way valve positioned to enable bodily fluid and other fluids to flow into the cavity through the inlet port, but not out of the cavity through the inlet port; and an outlet port comprising a gas permeable but liquid resistant membrane positioned to enable gas to be expelled from the cavity through the outlet port, but to resist bodily fluid and other fluids being expelled from the cavity through the outlet port, the outlet port further comprising a second one-way valve positioned to enable gas to be expelled from the cavity through the outlet port, but not into the cavity through the outlet port.

In some embodiments, the device further comprises an inlet connector releasably coupleable to the inlet port, the inlet connector comprising a first annular protrusion sized to couple in a substantially fluid-tight manner to a second annular protrusion of the inlet port, wherein the second annular protrusion is recessed within a cavity of the inlet port. In some embodiments, at least one of the first and second annular protrusions comprises a tapered surface sized to engage a mating surface of the other of the first and second annular protrusions to form a substantially fluid-tight seal. In some embodiments, the inlet connector further comprises a radially-extending protrusion positioned such that an external force applied to the radially-extending protrusion creates a moment about an axis of the first annular protrusion to enable a user of the fluid collection device to twist the inlet connector with respect to the inlet port. In some embodiments, the radially-extending protrusion extends in a radial direction a distance from the axis of the first annular protrusion that is at least 1.5 times a distance an outer surface of the first annular protrusion extends radially from the axis of the first annular protrusion. In some embodiments, the inlet connector further comprises a tubing interface comprising a plurality of annular tapered surfaces each sized to couple in a fluid-tight manner with flexible tubing of a different inner diameter. In some embodiments, the first one-way valve comprises a duckbill valve. In some embodiments, the liquid resistant membrane is positioned proximally to the second one-way valve with respect to the cavity. In some embodiments, the outlet port comprises an opening through which gas from within the cavity may be expelled to an atmosphere, the opening comprising a non-uniform end surface sized to enable a human finger to be placed on the end surface without forming a fluid-tight seal between the finger and the end surface. In some embodiments, the non-uniform end surface comprises an annular surface having one or more recessed portions. In some embodiments, the compressible resilient body comprises a bulb, wherein a shape memory of the bulb generates a vacuum within the cavity after the bulb has been compressed by an external force.

According to some embodiments, an aspiration and fluid collection device for collecting bodily fluid and other fluids comprises a body comprising a cavity for collection of these fluids; an inlet port in fluid communication with the cavity of the body; and an inlet connector releasably coupleable to the inlet port, the inlet connector comprising a first annular protrusion sized to couple in a substantially fluid-tight manner to a second annular protrusion of the inlet port, wherein the second annular protrusion is recessed within a cavity of the inlet port, wherein at least one of the first and second annular protrusions comprises a tapered surface sized to engage a mating surface of the other of the first and second annular protrusions to form a substantially fluid-tight seal.

In some embodiments, the inlet connector further comprises a tubing interface comprising a plurality of annular tapered surfaces each sized to couple in a fluid-tight manner with flexible tubing of a different inner diameter. In some embodiments, the body comprises a collapsible bulb comprising a flexible, resilient material. In some embodiments, the inlet port comprises a first one-way valve positioned to enable bodily fluid and other fluids to flow into the cavity through the inlet port, but not out of the cavity through the inlet port; wherein the fluid collection device further comprises an outlet port comprising a gas permeable but liquid resistant membrane positioned to enable gas to be expelled from the cavity through the outlet port, but to resist bodily fluid and other fluids being expelled from the cavity through the outlet port, the outlet port further comprising a second one-way valve positioned to enable gas to be expelled from the cavity through the outlet port, but not into the cavity through the outlet port. In some embodiments, the inlet connector further comprises a radially-extending protrusion positioned such that an external force applied to the radially-extending protrusion creates a moment about an axis of the first annular protrusion to enable a user of the fluid collection device to twist the inlet connector with respect to the inlet port. In some embodiments, the radially-extending protrusion extends in a radial direction a distance from the axis of the first annular protrusion that is at least 1.5 times a distance an outer surface of the first annular protrusion extends radially from the axis of the first annular protrusion.

According to some embodiments, a method of collecting bodily fluid and other fluids using an aspiration and fluid collection device comprises coupling a flexible tube in a substantially fluid-tight manner to an inlet connector; inserting a first annular protrusion of the inlet connector into a cavity of an inlet port of the fluid collection device; twisting the first annular protrusion with respect to the inlet port to generate a substantially fluid-tight seal between the first annular protrusion and a second annular protrusion of the inlet port, the second annular protrusion recessed within the cavity of the inlet port; compressing a resilient body of the fluid collection device such that an inner cavity of the body is reduced in volume; expelling gas from within the inner cavity through an outlet port, wherein the outlet port comprises a gas-permeable but substantially liquid impermeable barrier; and collecting bodily fluid and other fluids within the inner cavity of the body, the bodily fluid and other fluids caused to be transferred from the tube, through the inlet connector, and through the inlet port at least partially by a vacuum generated by the compressed resilient body.

In some embodiments, the flexible tube is coupled to at least one of a plurality of annular tapered surfaces of the inlet connector, each tapered surface sized to couple in a fluid-tight manner with flexible tubing of a different inner diameter. In some embodiments, the method further comprises stopping gas from being drawn into the inner cavity of the body through the outlet port using a one-way valve.

According to some embodiments, a fluid container for collecting bodily fluid and other fluids drained from a patient comprises a resilient body defining an interior cavity and capable of being compressed to reduce a volume of the interior cavity; an input port having: an input receptacle configured to form a liquid tight seal with a first end of a tube having a second end disposed on the patient to provide fluid communication from the patient to the container; a one way valve for allowing fluid to flow into and not out of the interior cavity; at output port having: an output receptacle; a one way valve for allowing fluid to flow out of and not into the inferior cavity; a gas permeable and liquid non-permeable member for allowing gas to flow through the output port and not fluid.

In some embodiments, the input receptacle is a recess formed in an upper portion of the body having a cylindrical configuration, and the recess of the input receptacle is sized to receive a tube connectable to a patient. In some embodiments, the gas permeable and liquid non-permeable member is a hydrophobic material. In some embodiments, the input receptacle has a cylindrical hole configuration. In some embodiments, the output receptacle is configured to provide an airtight seal with a line to a vacuum source.

For purposes of this summary, certain aspects, advantages, and novel features of the disclosure are described herein. It is to be understood that not necessarily all such advantages may be achieved in accordance with any particular embodiment of the disclosure. Thus, for example, those skilled in the art will recognize that the inventions disclosed herein may be embodied or carried out in a manner that achieves one advantage or group of advantages as taught herein without necessarily achieving other advantages as may be taught or suggested herein.

### BRIEF DESCRIPTION OF THE DRAWINGS

The foregoing and other features, aspects, and advantages of the present disclosure are described in detail below with reference to the drawings of various embodiments, which are intended to illustrate and not to limit the inventions disclosed herein.

FIG. 1A is a front view of an embodiment of an aspiration and fluid collection device.

FIG. 1B is a cross-sectional view of the device of FIG. 1A.

FIG. 1C is an exploded view of the device of FIG. 1A.

FIG. 2 is a cross-sectional view of an embodiment of an inlet and outlet interface or junction block of an aspiration and fluid collection device.

FIG. 3 is a cross-sectional view of an embodiment of an inlet connector of an aspiration and fluid collection device.

### DETAILED DESCRIPTION OF THE EMBODIMENTS

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

The disclosure herein provides systems, methods, and devices for portable suction and collection of bodily fluid and other fluids. A portable fluid suction and collection device may be desirable, for example, for use with patients that undergo abdomoniplasty, mastectomy and nasal procedures, such as rhinoplasty. For a period of time after a surgical procedure such as rhinoplasty, a patient may produce excess bodily fluid and other fluids, such as blood, which need to be collected and disposed of. It can be advantageous to have a portable device to perform such collection to enable a patient to be mobile and not restricted to a hospital or other medical facility during his or her recovery.

In some embodiments, it can be advantageous to not only passively collect bodily fluid and other fluids (for example, using gravity), but to actively collect those fluids, such as by generating a suction, vacuum, or aspiration force that actively causes the fluids to be sucked out of the patient and transferred to the collection device. In order to make such a device easy to use and easily portable, it can be advantageous to combine the suction or aspiration functions with the collection functions. Accordingly, it can be advantageous to have a single device that provides both a vacuum or suction force and also provides for collection of the excess fluids. In some embodiments, it is desirable to have a self-powered (for example, not electrically-powered) suction and collection device, so that a user does not need to worry about running out of power or safety concerns. For example, in some embodiments, suction is generated by compressing a resilient body, which generates a vacuum when it tries to re-expand to its original configuration. In some embodiments, however, an electrically-powered vacuum or suction source may be utilized.

An aspiration and fluid collection device in some embodiments comprises a resilient container such as a collapsible bulb having a cavity for collection of bodily fluid and other fluids. The container may have a volume of at least about 50 cc or 100 cc but generally less than about 1,000 cc and in many embodiments, less than about 500 cc or 250 cc. The device further comprises a removable cap having an inlet port and an outlet port each comprising a one-way valve. In some embodiments, the inlet port comprises a one-way valve positioned to enable flow of bodily fluids and other fluids or gases into the cavity of the bulb but not out of the cavity. Further, the outlet port comprises a one-way valve positioned to enable gas to flow outward from the cavity of the bulb but not inward to the cavity. With such a configuration, the fluid collection device can be configured to enable a user of the device to squeeze the bulb, reducing a volume of the cavity, and expelling or exhausting gas from within the cavity out through the one-way valve of the outlet port. The resiliency of the bulb material then causes the bulb to try to re-expand to its original shape or configuration, thus generating a vacuum or suction force through the one-way valve of the inlet port. This vacuum or suction force can be utilized to cause bodily fluid and other fluids to be sucked through a tube connected to a patient, through the inlet port, and into the cavity of the body.

In some embodiments, the fluid suction and collection device can be configured to retain collected fluids in the cavity of the body for disposal. In some embodiments, the fluid suction and collection device or system is a disposable unit that is intended to be disposed of after a single use. A single use in some embodiments is defined as collecting a volume of bodily fluid and other fluids that fills or substantially fills the cavity of the bulb, or at least fills the cavity of the bulb to a predetermined percentage of the volume of the cavity. In some embodiments, a single use is defined as a use for a predetermined amount of time.

The outlet port of the fluid collection and suction device in some embodiments comprises a filter or other material that is gas permeable but resistant to liquids or liquid non-permeable. In some embodiments, such filter or material may comprise a hydrophobic material. It can be advantageous to have such a material to, for example, enable gas to be expelled through the outlet port when a user squeezes or compresses the bulb, without also expelling the collected bodily fluid and other fluids out through the outlet port. Accordingly, including such a material can make a fluid collection device easier to use, less messy, and safer, because collected bodily fluid and other fluids, which may be considered a hazardous material, will not be expelled out of the device. In some embodiments, the outlet port comprises a nonuniform external surface that enables a user to place his or her finger over the outlet port while compressing the bulb without forming a seal over the outlet port.

A fluid suction and collection device comprises, in some embodiments, an inlet connector removably coupled to the inlet port. The inlet connector also can be configured to be coupled to tubing, such as medical grade silicone tubing. The inlet connector may make a fluid suction and collection device more convenient and easier to use, because a user can easily separate the inlet connector from the fluid collection device to enable changing to a new or fresh fluid collection device, without removing the tubing and related fluid collection structure that may be attached to the patient, and that would be more difficult—and potentially painful—to remove. For example, the tubing and related fluid collection structure may be positioned within a surgical site such as the patient's nasal cavity, and may be painful to remove, and also difficult to reinstall. In some embodiments, the tubing and other devices connected to the patient for initial collection of bodily fluid and other fluids from the nasal cavity or other post-operative wound, may comprise one or more of the embodiments illustrated and described in U.S. Patent Application Publication No. 2013/0331805 A1, entitled MEDICAL SUCTION SYSTEM AND DISPOSABLE CONTAINER, which is hereby incorporated by reference herein in its entirety.

In some embodiments, the inlet connector comprises a protrusion configured to couple in a fluid tight and/or airtight manner with a mating protrusion and/or recess of the inlet port. In some embodiments, the protrusion of the inlet connector and/or the inlet port comprises a tapered surface sized to couple with a mating surface of other of the inlet connector or inlet port. In some embodiments, the inlet connector comprises one or more handles or wings radially extending therefrom that enable a user to easily twist the inlet connector about a longitudinal axis of the connector as the connector engages or disengages the inlet port. Such twisting motion can make it easier to form a liquid tight and/or air tight seal with the inlet port, thus eliminating or reducing leakage around the inlet connector/inlet port interface. It can be advantageous to have such a liquid tight and/or air tight seal to enable the full or substantially all of the negative pressure or suction force created by the compressed bulb to be applied to the remote region of the patient from which bodily fluid and other fluids are being retrieved, such as the patient's nasal cavity or wound, as opposed to the suction force leaking through the connector junction.

In some embodiments, the inlet port comprises a recessed cavity or protrusion configured to couple with the inlet connector to generate the liquid tight or air tight connection. By making the protrusion or cavity recessed, this can be advantageous to reduce or eliminate splashing or spilling when disconnecting the inlet connector from the inlet port. Accordingly, such a recessed design can be safer, because bodily fluid and other fluids, which may be considered a hazardous material, will not splash or be ejected from the inlet connector or port when the inlet connector is removed from the inlet port.

In some embodiments, the inlet connector comprises a multi-use design configured to couple with a plurality of sizes of flexible tubing. For example, in some embodiments, the inlet connector comprises a protrusion having a plurality of tapered surfaces, each tapered surface having a different maximum outer diameter configured to engage a different size of tubing.

In some embodiments, the hydrophobic filter membrane or material of the outlet port comprises microbial properties. The filtering and/or microbial properties of this filter may be advantageous to enable gas expelled out of the outlet port to not comprise potential contaminants or offensive odors.

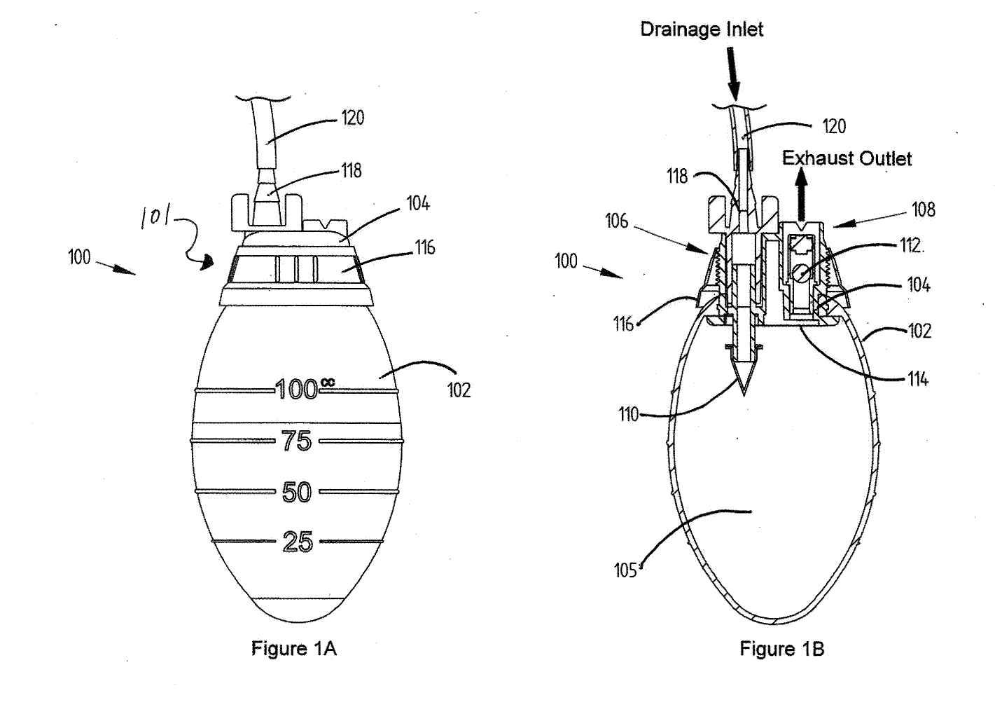

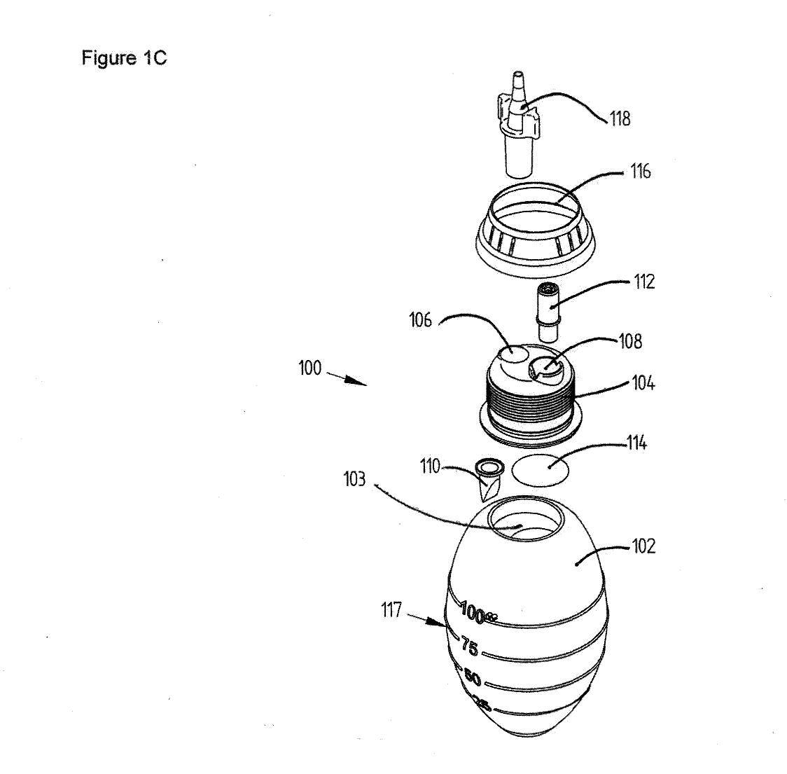

Referring now to the figures, FIGS. 1A-1C illustrate an embodiment of an aspiration and fluid collection device 100. FIG. 1A is a side view, FIG. 1B is a cross-sectional view, and FIG. 1C is an exploded view. The fluid collection device 100 comprises a rounded bulb 102 having a cavity 105, closed by a cap 101 which may be removably mounted or permanently bonded to the bulb 102. The cap 101 comprises a junction block 104, an inlet connector 118, and a retainer ring 116 among additional features discussed below.

The inlet connector is removably coupled to both the junction block 104 and a tube 120. In this embodiment, the tube 120 may, for example, lead to a device or system for collecting fluid at a surgical site, such as a nasal cavity or surgical wound of a patient. The tube 120 can be configured to transfer bodily fluid and other fluids from the surgical site to the inlet connector 118, enabling the bodily fluid and other fluids to pass through the inlet port 106 of the junction block 104, and the one-way valve 110, and then into the cavity 105 of the bulb 102. In this embodiment, a single junction block 104 comprises the inlet port 106 and outlet port 108 features. In other embodiments, however, the inlet port and outlet port may be positioned elsewhere and do not necessarily need to be part of the same junction block 104. Further, one or more of the ports may be integrated into the bulb 102, and there may be no need for a junction block 104.

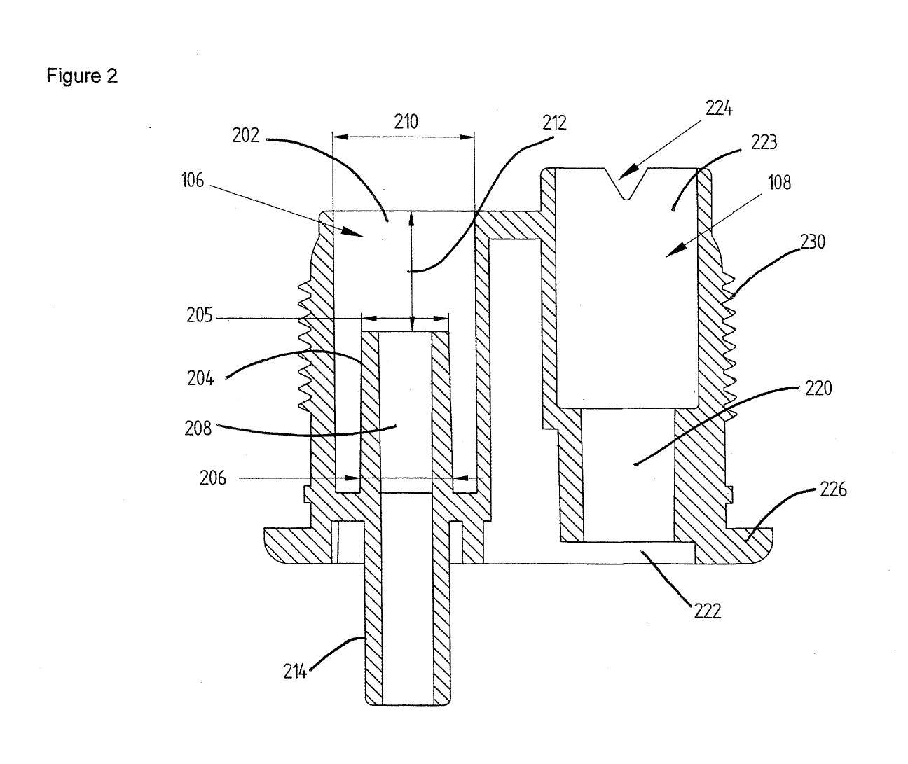

The junction block 104 comprises a radial protrusion 226, as shown in FIG. 2. A retainer 116 is configured to engage a thread 230 of the junction block 104, enabling the junction block 104 to be held in place in relation to the bulb 102. In some embodiments, an edge of an opening 103 of the bulb 102 is captured and/or compressed between the retainer 116 and the radial protrusion 226. Accordingly, the junction block 104 can be held in place with respect to the bulb 102, and a fluid tight and/or air tight seal may be made between the junction block 104 and the bulb 102. It can be desirable to have a fluid tight and/or air tight seal between the bulb 102 and the junction block 104 to enable more efficient operation of the suction and fluid collection device, by limiting loss of vacuum or negative pressure through any leaks.

As can be seen in FIG. 1B, the inlet port 106 comprises a one-way valve 110 position to enable gas and/or fluid to enter the cavity 105 through the inlet port 106, but not to exit the cavity 105 through the inlet port 106. In this embodiment, the one-way valve 110 comprises a duckbill style valve. However, in other embodiments, the one-way valve 110 may comprise a variety of other valve designs.

The outlet port 108 comprises another one-way valve 112. The one-way valve 112 is positioned such that gas can be exhausted out of the cavity 105 through the outlet port 108, but gas or fluid cannot reenter the cavity 105 through the outlet port 108. This can be advantageous to enable any negative pressure or vacuum or suction force generated by the resilient bulb 102 to affect only the inlet port 106, to provide suction at the surgical site or other location from which fluid is being collected. In this embodiment, the one-way valve 112 comprises a ball check style valve. However, as with the other one-way valve 110, a variety of other one-way valve designs may be utilized to perform the functions described herein.

The outlet port 108 further comprises a hydrophobic filter 114, as can be seen in FIG. 1B and the exploded view of FIG. 1C. The hydrophobic filter 114 is coupled to the junction block 104 within a cavity 222 as illustrated in FIG. 2. The hydrophobic filter 114 can be, in some embodiments, attached to the junction block 104 using adhesives, a friction fit, interference fit, mechanical fasteners, and/or the like.

As can be seen in FIG. 1C, the bulb 102 further comprises a plurality of volume markings 117 positioned around an outside surface of the bulb 102. Such markings can be desirable to enable a user of the device to determine how much fluid has been collected. In some embodiments, the bulb 102 comprises a clear or transparent or semitransparent material to enable a user to see the level of fluid within the device. In some embodiments, the bulb 102 comprises a rubber, silicone, or other compliant and/or resilient material. Further, although in this embodiment the bulb 102 comprises a rounded or egg-shape, various other embodiments of the bulb may comprise various other shapes. For example, the bulb 102 may comprise a rectangular, square, thin elongate structure, and/or flask-shaped body. Some shapes may be desirable to enable the collection device to, for example, fit in a user's pocket.

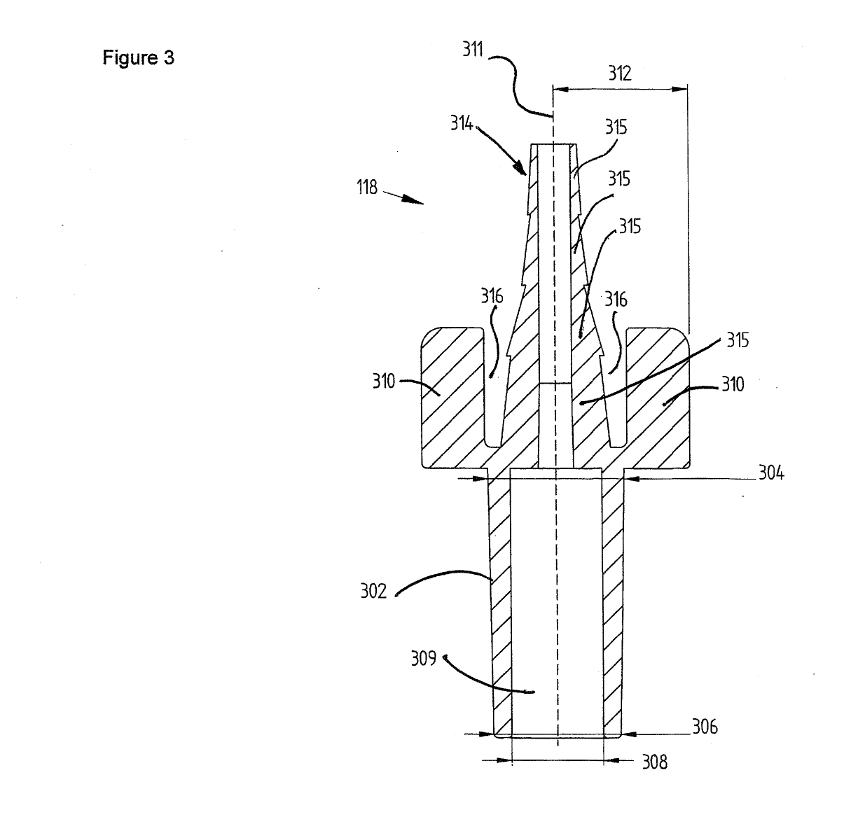

FIG. 2 is a cross-sectional view of the junction block 104 of the aspiration and fluid collection device 100 illustrated in FIGS. 1A-1C. FIG. 3 is a cross-sectional view of the inlet connector 118 of the aspiration and fluid collection device 100 shown in FIGS. 1A-1C. The junction block 104 comprises an axially extending annular protrusion 204 configured to mate with a complementary annular protrusion 302 of the inlet connector 118. The annular protrusions 204, 302 comprise tubular connectors which can be sized and configured to mate in a generally or substantially fluid tight and/or air tight arrangement. For example, in this embodiment, an outer surface of the annular portion 204 is tapered, with a smaller or minimum diameter 205 at a distal end, and a larger or maximum diameter 206 at a proximal end. The annular protrusion 302 of the inlet connector 118 comprises a cavity 309 having an inner diameter 308 that is sized concentrically to couple with the tapered outer surface of the annular portion 204. For example, in some embodiments, the inner diameter 308 is approximately 6.5 mm, and the outer surface of the annular protrusion 204 tapers from a minimum diameter 205 of 6.2 mm to a maximum diameter 206 of approximately 6.6 mm. Accordingly, the inlet connector 118 can be positioned over the annular protrusion 204 and twisted into place or pressed into place to form a press fit between the annular protrusion 204 and the cavity 309 of the annular protrusion 302. Accordingly, a fluid tight and/or air tight seal may be easily created by a user of the device. Although in this embodiment the cavity 309 comprises an inner diameter 308 of approximately 6.5 mm, other embodiments may comprise different inner diameters 308. For example, some embodiments may comprise an inner diameter 308 of at least, no more than, or approximately 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, or 10.0 mm. Further, the annular protrusion 204 may comprise a tapered outer surface of a size sufficient to couple with the inner diameter 308 in a fluid tight or air tight manner.

Although in this embodiment the outer surface of the annular protrusion 204 is tapered in design to mate with a non-tapered cavity 309 of the annular portion 302, it will be clear to one of skill in the art that a variety of other configurations may be utilized to achieve a similar fluid-tight and/or airtight connection. For example, the cavity 309 of the inlet connector 118 may be tapered, and the outer surface of the annular protrusion 204 may be straight. As another example, the outer surface of the annular protrusion 302 may be configured to generate the fluid tight or gas tight seal with the annular protrusion 204 and/or cavity 202. As another example, an O-ring or gasket may be utilized to generate the fluid tight or air tight seal.

In this embodiment, the tubular protrusion 204 is recessed within cavity 202. Such a design may be advantageous to limit spillage or splashing when a user is removing the inlet connector 118 from the tubular protrusion 204. The wall of the cavity 202 can act as a splash shield or spill shield when removing and/or inserting the inlet connector 118. In this embodiment, the cavity 202 comprises an annular cavity positioned concentrically with the tubular protrusion 204. However, in other embodiments, the cavity 202 may be other shapes. Further, in this embodiment, the annular protrusion 204 is recessed within the cavity 202 by a distance 212 of approximately 8.6 mm. However, in other embodiments, the distance 212 may be a variety of other values, for example, at least, no more than, or approximately 5.0 mm, 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.0 mm, 12.5 mm, 13.0 mm, 13.5 mm, 14.0 mm, 14.5 mm, or 15.0 mm. Further, in this embodiment, the cavity 202 comprises a diameter 210 of approximately 10 mm. However, in other embodiments, the diameter 210 may be other values, for example, at least, no more than, or approximately 5.0 mm, 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm, 18.5 mm, 19.0 mm, 19.5 mm, or 20.0 mm.

The junction block 104 further comprises a tubular protrusion 214 extending downwardly or away from the annular protrusion 204. The protrusion 214 can be configured and sized to mate with the one-way valve 110 depicted in FIG. 1C.

The outlet port 108 of the junction block 104 comprises an upper cavity 223, a lower cavity 222, and a lumen 220 passing therebetween. In some embodiments, the upper cavity 223 and/or lumen 220 are sized and configured to mate with a one-way valve, such as the one-way valve 112 illustrated in FIGS. 1B and 1C. Although in this embodiment the outlet port 108 comprises concentric annular portions configured to mate with the one-way valve 112, various other embodiments may comprise various other designs to enable coupling with a differently designed one-way valve.

In this embodiment, a distal surface of the upper cavity 223 comprises one or more vents such as at least one or two opposing voids or cutouts 224. The voids 224 cause the outlet port 108 to have an uneven or non-uniform outer distal surface, enabling a user of the device to place his or her finger over the outlet port 108 without completely sealing off the outlet port 108. Accordingly, by having a non-uniform outer surface, a user can compress the bulb 102, expelling gas out of the outlet port 108, while the user has his or her finger over the outlet port 108, which may be desirable to enable compression of the bulb 102 with a single hand. Although the voids 224 in this embodiment are V-shaped, various other non-uniform surface designs may be utilized. Further, in some embodiments, one or more separate vent holes or other opening into the side wall of outlet port 108 may be utilized, instead of a non-uniform distal surface.

The junction block 104 further comprises a thread 230 configured to mate with a thread of the retainer or cap 116. Although in this embodiment a thread is used, in other embodiments, various other fastening means may be used, such as adhesives, latches, friction, a snap-fit, twist fit and/or the like. In some embodiments, the junction block 104 is directly coupled to the bulb 102 without requiring the retainer or cap 116.

The inlet connector 118 illustrated in FIG. 3 comprises an annular tubular protrusion 302 sized and configured to fit within the cavity 202 of the junction block 104. In some embodiments, an outer surface of the annular protrusion 302 is tapered, such as to enable easier insertion and retraction of the inlet connector 118 to or from the cavity 202 (and/or to create a liquid tight or air tight seal with the cavity 202). In this embodiment, an outer surface of the tubular protrusion 302 comprises a minimum diameter 306 of approximately 9 mm, and a maximum diameter 304 of approximately 9.7 mm. However, in various other embodiments, the annular protrusion 302 may comprise an outer surface having different tapered diameters, a non-tapered diameter, a noncircular outer surface, such as a square, and/or the like.

The inlet connector 118 further comprises a tubing interface 314 comprising a plurality of annular tapered surfaces 315 forming a plurality of annular barbs configured to mate with different sized tubes. For example, each of the tapered surfaces 315 may comprise a different size configured to connect in a fluid tight and/or gas tight manner with a flexible tube of a different inner diameter.

The inlet connector 118 further comprises at least one or two or more handles or wings 310 extending radially. The handles or wings 310 can be configured to enable a user of the inlet connector 118 to more easily twist the connector while attaching or detaching the connector to or from the inlet port 106. In this embodiment, the handles or wings 310 extend radially a distance 312 from a longitudinal axis 311 approximately 9.5 mm. However, in various other embodiments, the handles or wings 310 may extend a smaller or greater distance, such as, for example, at least, no more than, or approximately 5.0 mm, 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm, 18.5 mm, 19.0 mm, 19.5 mm, or 20.0 mm. In some embodiments, the handles or wings 310 may extend in a radial direction a distance from the axis 311 that is at least 0.50, 0.75, 1.00, 1.25, 1.50, 1.75, 2.00, 2.25, 2.50, 2.75, or 3.00 times a distance an outer surface of the annular protrusion 302 extends radially from the axis 311. Further one or more handles or wings 310 may comprise various other designs or configurations, as long as they provide a protruding surface for a human finger to engage to help in twisting the inlet connector 118 about the longitudinal axis 311.

In some embodiments, a fluid suction and containment device comprises a resilient bulb configured to be compressible to generate a suction force (for example, the bulb 102). The device further comprises an interface portion disposed at a top end of the bulb, the interface portion comprising an inlet port and an output port (for example, the junction block 104). The inlet port comprises a one-way valve (for example, the one-way valve 110), in some embodiments a duckbill valve, and a double walled connector portion (for example, the cavity 202 and protrusion 204) configured to mate with a tubing connector (for example, the inlet connector 118). In some embodiments, the tubing connector comprises a stepped portion (for example, the tubing interface 314) that enables the tubing connector to connect to flexible tubing of a variety of inner diameters. In some embodiments, the tubing connector comprises one or more handles or wings (for example, the handles 310) protruding radially from the connector that are configured to enable a user of the device to rotate the connector while inserting or retracting the connector from the interface portion of the device, to make the connector easier to insert and/or retract and/or to enable creation of a tighter friction or press fit with the interface portion. In some embodiments, the connector comprises a tapered outer and/or inner annular surface (for example, a surface of the annular portion 302) configured to engage an annular portion of the interface portion of the device (for example, the cavity 202 and/or annular protrusion 204). In some embodiments, the inlet port of the interface portion comprises a recessed annular portion (for example, the annular protrusion 204) that is recessed within another annular region (for example, the cavity 202) having a larger diameter than the recessed portion. In some embodiments, such a recessed design can reduce a risk of splashing when the tubing connector is removed from the device. In some embodiments, the outlet port comprises one or more recesses (for example, the voids 224), in this embodiment a V-shaped recess, which can be advantageous to enable a user to place his or her finger over the outlet opening without completely sealing the opening. In some embodiments, the outlet port also comprises a one-way valve (for example, the one-way valve 112). In some embodiments, the device comprises a hydrophobic filter (for example, the filter 114) configured to enable air or gas to be expelled through the outlet port, but not liquid.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Although the present disclosure has been presented in the context of certain preferred embodiments and examples, it will be understood by those skilled in the art that the present inventions extend beyond the specifically disclosed embodiments to other alternative embodiments and/or uses of the inventions and obvious modifications and equivalents thereof. Additionally, the skilled artisan will recognize that any of the above-described methods can be carried out using any appropriate apparatus. Further, the disclosure herein of any particular feature, aspect, method, property, characteristic, quality, attribute, element, or the like in connection with an embodiment can be used in all other embodiments set forth herein. For all of the embodiments described herein the steps of the methods need not be performed sequentially. Thus, it is intended that the scope of the present disclosure should not be limited by the particular embodiments described above.

What is claimed is:

1. An aspiration and fluid collection device for collecting bodily fluid and other fluids, the device comprising:
a connector configured for connection to a compressible resilient body comprising a cavity for storage of collected bodily fluid and other fluids;
an inlet port comprising a first one-way valve positioned to enable bodily fluid and other fluids to flow into the cavity through the inlet port, but not out of the cavity through the inlet port; and
an outlet port comprising a gas permeable but liquid resistant membrane positioned to enable gas to be expelled from the cavity through the outlet port, but to resist bodily fluid and other fluids being expelled from the cavity through the outlet port,
the outlet port further comprising a second one-way valve positioned to enable gas to be expelled from the cavity through the outlet port, but not into the cavity through the outlet port.

2. The aspiration and fluid collection device of claim 1, further comprising an inlet connector releasably coupleable to the inlet port, the inlet connector comprising a first annular protrusion sized to couple in a substantially fluid-tight manner to a second annular protrusion of the inlet port, wherein the second annular protrusion is recessed within a cavity of the inlet port.

3. The aspiration and fluid collection device of claim 2, wherein at least one of the first and second annular protrusions comprises a tapered surface sized to engage a mating surface of the other of the first and second annular protrusions to form a substantially fluid-tight seal.

4. The aspiration and fluid collection device of claim 2, wherein the inlet connector further comprises a radially-extending protrusion positioned such that an external force applied to the radially-extending protrusion creates a moment about an axis of the first annular protrusion to enable a user of the fluid collection device to twist the inlet connector with respect to the inlet port.

5. The aspiration and fluid collection device of claim 4, wherein the radially-extending protrusion extends in a radial direction a distance from the axis of the first annular protrusion that is at least 1.5 times a distance an outer surface of the first annular protrusion extends radially from the axis of the first annular protrusion.

6. The aspiration and fluid collection device of claim 2, wherein the inlet connector further comprises a tubing interface comprising a plurality of annular tapered surfaces each sized to couple in a fluid-tight manner with flexible tubing of a different inner diameter.

7. The aspiration and fluid collection device of claim 1, wherein the first one-way valve comprises a duckbill valve.

8. The aspiration and fluid collection device of claim 1, wherein the liquid resistant membrane is positioned proximally to the second one-way valve with respect to the cavity.

9. The aspiration and fluid collection device of claim 1, wherein the outlet port comprises an opening through which gas from within the cavity may be expelled to an atmosphere, the opening comprising a non-uniform end surface sized to enable a human finger to be placed on the end surface without forming a fluid-tight seal between the finger and the end surface.

10. The aspiration and fluid collection device of claim 9, wherein the non-uniform end surface comprises an annular surface having one or more recessed portions.

11. The aspiration and fluid collection device of claim 1, wherein the compressible resilient body comprises a bulb, wherein a shape memory of the bulb generates a vacuum within the cavity after the bulb has been compressed by an external force.

12. An aspiration and fluid collection device for collecting bodily fluid and other fluids, the device comprising:
a body comprising a cavity for collection of bodily fluid and other fluids;
an inlet port in fluid communication with the cavity of the body; and
an inlet connector releasably coupleable to the inlet port, the inlet connector comprising a first annular protrusion sized to couple in a substantially fluid-tight manner to a second annular protrusion of the inlet port, wherein the second annular protrusion is recessed within a cavity of the inlet port,
wherein at least one of the first and second annular protrusions comprises a tapered surface sized to engage a mating surface of the other of the first and second annular protrusions to form a substantially fluid-tight seal.

13. The aspiration and fluid collection device of claim 12, wherein the inlet connector further comprises a tubing interface comprising a plurality of annular tapered surfaces each sized to couple in a fluid-tight manner with flexible tubing of a different inner diameter.

14. The aspiration and fluid collection device of claim 12, wherein the body comprises bulb comprising a flexible, resilient material.

15. The aspiration and fluid collection device of claim 12, wherein the inlet port comprises a first one-way valve positioned to enable bodily fluid and other fluids to flow into the cavity through the inlet port, but not out of the cavity through the inlet port; wherein the fluid collection device further comprises an outlet port comprising a gas permeable but liquid resistant membrane positioned to enable gas to be expelled from the cavity through the outlet port, but to resist bodily fluid and other fluids being expelled from the cavity through the outlet port, the outlet port further comprising a second one-way valve positioned to enable gas to be expelled from the cavity through the outlet port, but not into the cavity through the outlet port.

16. The aspiration and fluid collection device of claim 12, wherein the inlet connector further comprises a radially-extending protrusion positioned such that an external force applied to the radially-extending protrusion creates a moment about an axis of the first annular protrusion to enable a user of the fluid collection device to twist the inlet connector with respect to the inlet port.

17. The aspiration and fluid collection device of claim 16, wherein the radially-extending protrusion extends in a radial direction a distance from the axis of the first annular protrusion that is at least 1.5 times a distance an outer surface of the first annular protrusion extends radially from the axis of the first annular protrusion.

18. A method of collecting bodily fluid and other fluids using an aspiration and fluid collection device, the method comprising:
coupling a flexible tube in a substantially fluid-tight manner to an inlet connector;
inserting a first annular protrusion of the inlet connector into a cavity of an inlet port of the fluid collection device;
twisting the first annular protrusion with respect to the inlet port to generate a substantially fluid-tight seal between the first annular protrusion and a second annular protrusion of the inlet port, the second annular protrusion recessed within the cavity of the inlet port;
compressing a resilient body of the fluid collection device such that an inner cavity of the body is reduced in volume;
expelling gas from within the inner cavity through an outlet port, wherein the outlet port comprises a gas-permeable but substantially liquid impermeable barrier; and
collecting bodily fluid and other fluids within the inner cavity of the body, the bodily fluid and other fluids caused to be transferred from the tube, through the inlet connector, and through the inlet port at least partially by a vacuum generated by the compressed resilient body.

19. The method of claim 18, wherein the flexible tube is coupled to at least one of a plurality of annular tapered surfaces of the inlet connector, each tapered surface sized to couple in a fluid-tight manner with flexible tubing of a different inner diameter.

20. The method of claim 18, further comprising stopping gas from being drawn into the inner cavity of the body through the outlet port using a one-way valve.
